(19) United States
(12) Patent Application Publication
(10) Pub. No.: US 2023/0414609 A1
(43) Pub. Date: Dec. 28, 2023

(54) USES OF ORAI1-SPECIFIC CALCIUM CHANNELS IN TREATMENT OF ORAL CANCER

(71) Applicant: New York University, New York, NY (US)

(72) Inventors: Rodrigo Lacruz, New York, NY (US); Ga-Yeon Son, New York, NY (US)

(21) Appl. No.: 18/339,773

(22) Filed: Jun. 22, 2023

Related U.S. Application Data
(60) Provisional application No. 63/354,614, filed on Jun. 22, 2022.

(51) Int. Cl. A61K 31/497; A61P 35/00; A61K 31/44; A61K 31/433; A61K 31/415

(52) U.S. Cl. CPC A61K 31/497; A61P 35/00; A61K 31/44; A61K 31/433; A61K 31/415

(57) ABSTRACT

Provided are methods for treating oral cancer and inhibition allodynia. The methods involve administering to an individual who has oral cancer one or more agents that can inhibit ORAI Ca²⁺ channels.

### CROSS REFERENCE TO RELATED APPLICATIONS

This application claims priority to U.S. provisional application No. 63/354,614, filed Jun. 22, 2022, the entire disclosure of which is incorporated herein by reference.

### STATEMENT REGARDING FEDERALLY SPONSORED RESEARCH

This invention was made with government support under grant nos. DE025639, DE027679, and DE027981 awarded by the National Institutes of Health. The government has certain rights in the invention.

### BACKGROUND OF THE DISCLOSURE

Mutations in oncogenes remains one of the primary reasons for the onset of cancer, but as cancer progresses, other genes and their functions are affected, contributing to the transition to a more aggressive cancer ¹. During this oncogenetic transformation, the abnormal expression of genes coding for ion channels are emerging as important contributors in cancer development because these channels regulate ion concentration within cells affecting key functions such as proliferation, a hallmark of cancer ¹⁻⁵. Oral cancer, which are primarily squamous cell carcinomas, arise from oral epithelial dysplasia and commonly develop in the epithelial layer of the tongue. Oral cancer cells release nociceptive mediators that activate and sensitize primary afferent neurons, causing persistent pain that gets worse with disease progression. Early diagnosis and management of oral cancers are difficult, resulting in high morbidity and mortality. Treatment is primarily surgical causing speech difficulty affecting the quality of life. Identifying molecular biomarkers and processes underlying the initiation and progression of oral cancer are important to improve prognosis and treatment. Matrix metalloproteases (MMPs) such as MMP-1 are recognized as important markers in the transition from oral dysplasia to cancer degrading components of the extracellular matrix enabling the invasion of neoplastic cells into the underlying tissues ⁶⁻⁹

The highly specialized Ca²⁺ release activated Ca²⁺ (CRAC) channels, also known as store operated Ca²⁺ entry (SOCE), have been recently associated with various cancers ^{3,4}. Changes in Ca²⁺ concentration elicited by SOCE affect a wide range of cellular responses including gene expression and cell proliferation. SOCE is activated by a decrease in the concentration of Ca²⁺ stored in the endoplasmic reticulum (ER). This decline is recognized by the ER resident Ca²⁺ sensors proteins STIM1 and STIM2 which undergo oligomerization leading to the binding of the pore forming ORAI proteins (ORAI1-3) in the plasma membrane, to enable sustained Ca²⁺ influx ¹⁰⁻¹². Mutations in STIM1 and ORAI1 genes are linked to lung adenocarcinoma, colorectal, uterine and other cancers ^{1,3,4,13-15} while overexpression of SOCE elements is also associated with lung, liver, breast, renal, stomach and other cancers ¹⁶⁻¹⁸. However, there is an ongoing and unmet need to provide approaches for treatment or prophylaxis of oral cancers that involve SOCE. The present disclosure is pertinent to this need.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a fuller understanding of the nature and objects of the disclosure, reference should be made to the following detailed description taken in conjunction with the accompanying figures.

FIG. 1: (A) Heatmap contrast of average variance stabilizing transformation (VST) of ORAI1, ORAI2 and ORAI3 genes between normal (control: N=3 samples) and tongue cancer patients (N=10 samples). Statistical significance was analyzed using the Benjamini-Hochberg FDR (5%) and fold change of 1.5. Both FDR values and fold change were generated using DESeq2. (B-D) show haematoxylin and eosin (H&E) staining (left) and immunofluorescence staining of ORAI1 (right) in the boxed areas of tongue tissue sections showing normal (B), dysplastic (C) and invasive tumor (D) phenotypes. Nuclear staining by 4′,6-diamidino-2-phenylindole (DAPI) is shown in blue. Scale bar, 200 μm. (E) shows qRT-PCR analysis by the 2^{-ΔCT} method of ORAI1, ORAI2 and ORAI3 genes in human normal keratinocytes (HaCaT), dysplastic DOK cells and in the oral cancer cell lines HSC-3 and SCC-9. (F) shows measurements of [Ca²⁺]_{cyto} (shown as F340/F380 ratio) after SOCE stimulation with cyclopiazonic acid (CPA) and re-addition of 2 mM Ca²⁺ and quantification of SOCE corrected for baseline Ca²⁺ levels as Δpeak (F−F₀). (G) shows measurements of [Ca²⁺]_{cyto} after SOCE stimulation with CPA in HSC-3 cells in the presence or absence of the ORAI inhibitor synta-66 and quantification of SOCE corrected for baseline Ca²⁺ levels as Δpeak (F−F₀). (H) and (J) show qRT-PCR analyses of ORAI1 gene expression by the 2^{-ΔΔCT} method comparing control (scrambled shRNA) and shORAI1 (ORAI1 KD) HSC-3 cells (H) or CRISPR/Cas9 ORAI1 (ORAI1 KO) (J). (I) and (K) show [Ca²⁺]_{cyto} measurements comparing control HSC-3 cells compared to shORAI1 (ORAI1 KD) (I) or compared to CRISPR/Cas9 ORAI1 (ORAI1 KO) (K) (left). Quantification of SOCE corrected for baseline Ca²⁺ levels as Δpeak (F−F₀) (right). (L) and (M) show cell proliferation measured using MTS comparing control HSC-3 cells compared to shORAI1 (ORAI1 KD) (L) or compared to CRISPR/Cas9 ORAI1 (ORAI1 KO) (M).

FIG. 2: (A) shows qRT-PCR analysis of MMP1 gene expression in human keratinocytes (HaCaT), dysplastic DOK cells and in the oral cancer HSC-3 cell lines. (B) shows qRT-PCR analysis by the 2^{-ΔΔCT} method of MMP1 gene expression in CPA stimulated HSC-3 cells stimulated in the presence or absence of the ORAI inhibitor synta-66 or the NFAT blocker cyclosporine A (CsA). (C) shows qRT-PCR analysis of MMP1 gene expression in DOK cells overexpressing STIM1 and ORAI1 prior to and following the activation of SOCE with CPA. (D) is a schematic of the Boyden chambers and solutions used to quantify cell invasion. DOK cells overexpressing ORAI1 (O1) and STIM1 (S1) were seeded in the top chamber with 2% FBS. Media in the bottom chamber contained 10% FBS. (E) shows quantification of fold-change differences in the number of invading cells between control DOK cells (untransfected) and overexpressing STIM1 and ORAI1. (F) is a heatmap of log 2 fold-change of eight significantly (FDR<0.05) down-regulated MMP genes in the two contrasts indicated (control compared to CPA (+CPA) and CPA compared to synta-66 (+synta-66)) (N=4 biological replicates per group).

FIG. 3: (A) shows paw volume measurements in xenografted athymic mice rear paws using a Plethysmometer at 0, 3, 7, 10, and 14 days following the inoculation of control or shORAI1 (ORAI1 KD) HSC-3 cells. (B) shows quantification of paw volume in fixed H&E-stained paw tissue sections of each three control and shORAI1 (ORAL KD) HSC-3 cells inoculated mice. (C) shows representative images of H&E-stained tissue sections. Scale bar, 2 mm (upper images) and 200 μm (lower images). (D) and (E) show qRT-PCR analysis of ORAI1 (D) and MMP1 (E) gene expression in homogenized paw tissues from control and shORAI1 (ORAL KD) HSC-3 inoculated mice.

FIG. 4: (A) shows qRT-PCR analysis of Orai1 deletion in shOrai1 mouse oral cancer MOC-2 cells. (B) shows measurements of [Ca²⁺]_{cyto} after SOCE stimulation with CPA and re-addition of 2 mM Ca²⁺ in control or shOrai1 (Orai1 KD) MOC-2 cells (left), and quantification of SOCE corrected for baseline Ca²⁺ levels as Δpeak (F−F₀) (right). (C) is a schematic of the timeline used to assess allodynia in vivo. (D) and (E) show paw volume measured at the time indicated using a Plethysmometer in mice inoculated with wildtype MOC-2 cells (WT), shOrai1 MOC-2 cells and control (D), and quantification of tumor volume in H&E-stained paw tissue sections of control and shOrai1 (Orai1 KD) MOC-2 cells inoculated mice (E). (F) shows representative H&E-stained paw tissue sections. (G) shows quantification of withdrawal threshold in mice stimulated using a von Frey filament. (H) shows quantification of paw withdrawal latency in mice stimulated using a heat source.

FIG. 5: (A) shows Ca²⁺]_{cyto} measurements in mouse trigeminal neurons (TG) stimulated with the supernatant collected from control or CRISPR/Cas9 ORAI1 (ORAI1 KO) HSC-3 cells (left), and quantification of SOCE corrected for baseline Ca²⁺ levels as Δpeak (F−F₀) (right). (B) shows quantification of MMP1 by ELISA of the supernatant collected from control or CRISPR/Cas9 ORAI1 (ORAI1 KO) HSC-3 cells. (C) shows representative traces (top) and traces at 50 pA (middle) and 100 pA (bottom) in TG in the absence (black) or presence (red) of activated MMP1 stimulation. (D) shows quantification of the number current-evoked action potentials in response to 0-100 pA of injected current. (E) shows quantification of the rheobase in TG stimulating with/without activated MMP1 peptide. (F) shows a summary of resting membrane potential (RMP) in TG stimulating with/without activated MMP1 peptide.

### SUMMARY

The present disclosure provides approaches for prophylaxis or treatment of oral cancer and allodynia associated with the oral cancer. The method comprises administering to an individual who has oral cancer a composition comprising a calcium channel blocker. The calcium channel blocker may be specific for ORAI Ca²⁺ channels. Representative calcium channel blockers include synta66, BTP-2 (YM-58483), CM-2489, CM-4620, GSK-5498A, and GSK-7975A. Administering the composition comprising the calcium channel blocker inhibits growth of the oral cancer, or inhibits development of allodynia in the individual, or produces a combination thereof. Inhibiting the growth of the oral cancer may comprise inhibiting transformation of oral keratinocytes into a squamous cell carcinoma.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The role of SOCE in oral cancer pain has not been investigated despite reports implicating ORAI1 in nociception ^{19,20}. To address the role of CRAC channels in the transformation of normal oral keratinocytes to squamous cell carcinoma and its possible role in pain, we analyzed SOCE and Ca²⁺ dynamics in human and murine oral cancer cell lines and in dysplastic oral keratinocytes. We show that SOCE dominates the ability of these cells to uptake Ca²⁺ and that SOCE stimulation results in increased expression of MMP-1 and several other MMPs, even in dysplastic cells, upregulating the expression of these biomarkers. Inoculation of oral cancer cells lacking ORAI1 in mice not only reduced tumor growth, but it also decreased allodynia. Trigeminal neurons stimulated with the supernatant of oral cancer cells lacking ORAI1 evoked decreased excitation. These results suggest that SOCE has the potential to be an important target in the treatment of oral cancer and associated pain.

In an aspect, the present disclosure provides for use of compositions in treating cancer, pain associated with cancer, allodynia, or a combination thereof.

A composition administered to an individual in need thereof may comprise one or more calcium channel blockers. In various examples, the calcium channel blockers are specific for ORAI Ca²⁺ channels. Examples of calcium channel blockers that can be used in approaches of this disclosure include but are not necessarily limited to synta66, BTP-2 (YM-58483), CM-2489, Zegocractin (CM-4620), GSK-5498A, GSK-7975A, and the like, and combinations thereof. Synta66, BTP-2 (YM-58483), CM-2489, and Zegocractin (CM-4620) are considered to be potent blockers of SOCs.

The disclosure includes use of a composition comprising an ORAI1 channel blocker wherein the composition comprises any suitable nanoparticle delivery system. In embodiments, the disclosure includes use of the ORAI1 blocker CM-4620 (MedChemExpress cat #HY-101942) delivered as a nanoparticle formulation. CM-4620 is also referred to as Zegocractin. CM-4620 is an FDA approved drug used for use in treatment of pancreatitis, but has not been used for oral cancer. Thus, in embodiments, an individual to whom a composition of this disclosure is administered does not have pancreatic cancer.

A described composition may comprise additional components. For example, the composition may comprise a buffer solution suitable for administration to an individual (e.g., a mammal such as, for example, a human or a non-human). The buffer solution may be a pharmaceutically acceptable carrier.

A described composition may include one or more standard pharmaceutically acceptable carrier(s). Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water for injection, physiological saline, vegetable oil, alcohol, and the like, and combinations thereof. Further, injections may contain stabilizers, solubilizers, suspending agents, emulsifiers, soothing agents, buffers, preservatives, and the like. Injections may be sterilized in the final formulation step or prepared by sterile procedure. The composition may also be formulated into a sterile solid preparation, for example, by freeze-drying, and can be used after sterilized or dissolved in sterile injectable water or other sterile diluent(s) immediately before use. Non-limiting examples of pharmaceutically acceptable carriers can be found in: Remington: The Science and Practice of Pharmacy (2012) 22nd Edition, Philadelphia, PA. Lippincott Williams & Wilkins.

Acceptable carriers, excipients, or stabilizers are nontoxic to recipients at the dosages and concentrations employed, and include, but are not limited to, buffers such as, for example, phosphate, citrate, histidine and other organic acids; antioxidants including, but not limited to, ascorbic acid and methionine; preservatives (such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids, such as, for example, glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, polyethylene glycol (PEG) and the like. In an embodiment, the pharmaceutical composition may comprise buffer components and stabilizers, including, but not limited to, sucrose, polysorbate 20, NaCl, KCl, sodium acetate, sodium phosphate, arginine, lysine, trehalose, glycerol, and maltose.

The compositions may be administered by various methods. Administration can be carried out using any suitable route of administration known in the art. For example, the compositions may be administered via intravenous, intramuscular, intraperitoneal, intracerobrospinal, subcutaneous, intra-articular, intrasynovial, oral, or topical. The composition may be administered locally to the cancer, or distally. In embodiments the composition is introduced into the oral cavity. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated. In various examples, the injections can be directly into a cancer site (e.g., an oral tumor).

In one non-limiting approach, the disclosure includes analyzing cell invasion in the murine oral cancer cell line MOC-2 cells using Boyden chambers and treating the cells with CM-4620 in titrated concentrations, which may include any concentration that is 1 nM to 100 mM. In non-limiting examples, the titrations include 10 nM, 100 nM, 300 nM, and 1 μM concentrations of CM-4620 in a suitable volume. The disclosure includes administering an effective amount of CM-4620 determined at least in part on the titration analysis to inhibit cancer cell invasion. The disclosure includes administering CM-4620 to two groups of male and female wild type (C57BL/6J, strain #0006644) mice with MOC-2 cells (10000 cells in 20 μL) in the paw as a xenograft model of oral cancer. The paw model is used instead of introducing cancer cells into the tongue to more conveniently visualize and measure changes in tumor growth, and serves as a proof-of-concept model to validate the described approach. A baseline measurement is made using a Plethysmometer prior to treatment. Tumor growth is measured every 5 days. When the Plethysmometer measurements of the injected paw show significant changes from baseline (e.g. ˜0.2 ml increase from baseline or approximately 2 weeks after inoculation), a clear indication of tumor development, one group of mice receives CM4260 encapsulated within silica nanoparticles referred to in the art as “smart mesoporous balls (SMBs).” The other group of mice receives SMBs containing saline. Tumor growth is monitored for 3 weeks using a Plethysmometer. At the end of this period, paw tissues are dissected and decalcified to examine the histology of the tumors and to perform measurements of the area occupied by the tumor. An expected outcome of this process is inhibition of tumor growth in the paw model, which will be considered extendable to use for treating oral cancer and allodynia.

In examples, a subject in need of treatment is administered a therapeutically effective amount of a composition of the present disclosure, including but not necessarily limited to CM-4620, which may be provided as a nanoparticle formulation. In embodiments, a therapeutically effective amount of a described composition is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models as discussed above. As discussed above, an animal model can be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, a therapeutically effective amount of a described composition inhibits the growth of oral cancer, inhibits metastasis from an oral cancer tumor, inhibits oral cancer cell invasions, reduces or inhibits the development of allodynia associated with cancer, or a combination thereof. In embodiments the oral cancer that is treated according to a method of this disclosure is a squamous cell carcinoma.

The compositions may be administered to an individual or subject in need of treatment such as a human or a non-human animal. An individual in need of treatment may be a human. In other examples, the individual is a non-human mammal or other animal. Non-limiting examples of non-human mammals include cows, horses, pigs, mice, rats, rabbits, cats, dogs, or other agricultural mammals, pets, or service animals, and the like.

In various examples, the method may comprise introducing cells that do not express ORAI1 into an individual. The cells may be introduced directly into a site comprising malignant cancer cells (e.g., an oral tumor).

As an alternative or in addition to targeting calcium channels, the disclosure also includes targeting the gene encoding ORAI1 in an individual who has cancer, such as an oral cancer. Thus, the disclosure includes disrupting expression of the ORAI1 gene. This can be achieved, for example, using guide-directed RNA nucleases, TALONS, zinc fingers, transposon-bases systems, and other designer nucleases. The expression of the ORAI1 gene can also be inhibited using RNAi methods, including but not necessarily limited to siRNA and shRNA approaches.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following example is presented to illustrate the present disclosure. It is not intended to be limiting in any matter.

### Example 1

Results

ORAI1, ORAI2 and STIM2 are Upregulated in Tumors of Oral Cancer Patients

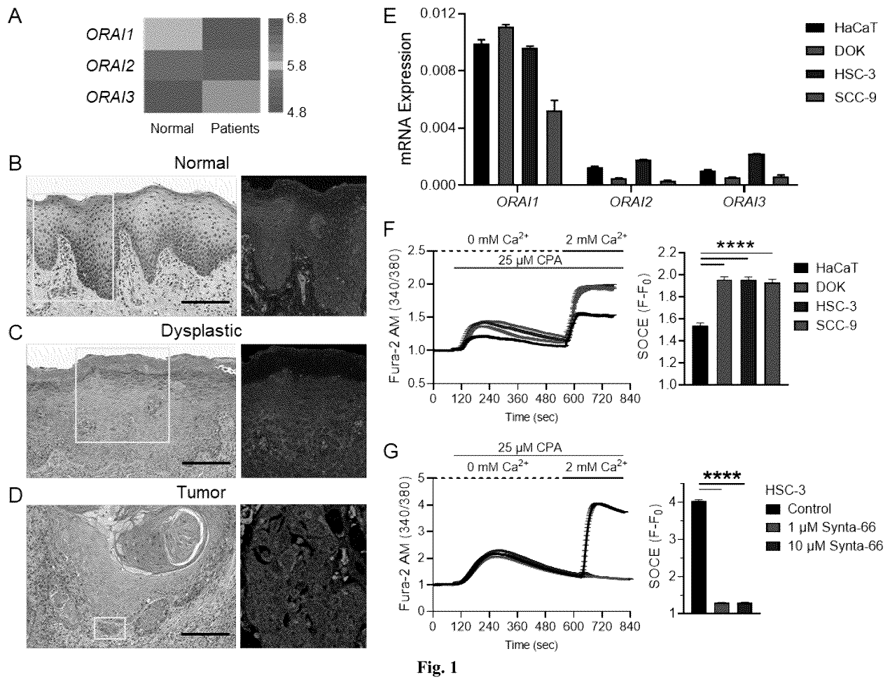

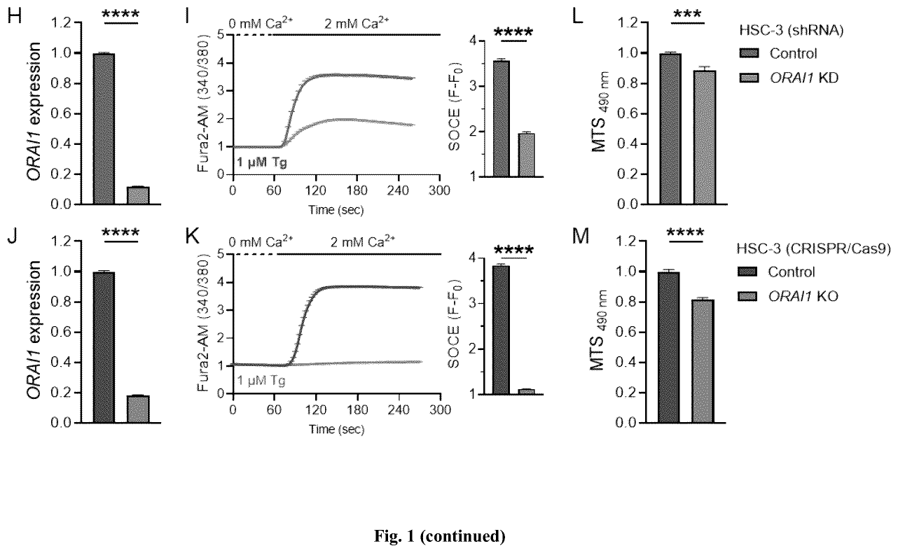

To address whether the molecular components of SOCE are differentially expressed in oral cancer tumors, we analyzed the expression of ORAI 1-3 genes in tongue tumors of oral cancer patients and healthy donors by RNA sequencing (RNA-seq). This analysis exploited data we had reported previously but had not carried out detailed analysis of these specific genes ²¹. Samples were obtained from patients with metastatic cancer or non-metastatic tumors and from normal/healthy patients ²¹. These data showed that ORAI1 and ORAI2 were significantly up-regulated in the tumor samples relative to those from normal healthy patients (FIG. 1A). Immunofluorescence images revealed that ORAI1 is expressed in the basal region of human tongue of normal tissue and oral dysplasia, being also expressed in metastatic tumors (FIG. 1, B-D). ORAI1 proteins are also expressed in the epithelial layer of murine tongue.

Ca²⁺ Influx in Oral Cancer Cells is Controlled by SOCE

To investigate SOCE in oral cancer cells, we first used qRT-PCR analysis to analyze the expression of ORAI1-3 in the human oral squamous cell carcinoma (OSCC) cell lines HSC-3 and SCC-9, in the dysplastic oral keratinocytes DOK cells, and in spontaneously transformed normal keratinocytes HaCaT cells. We showed that all cells expressed the three ORAI1-3 genes (FIG. 1E). Next, we tested whether these cells had functional SOCE by stimulating them with cyclopiazonic acid (CPA), a reversible blocker of the sarco-endoplasmic reticulum Ca²⁺ ATPase (SERCA) pump that passively depletes the ER Ca²⁺ stores inducing STIM1/ORAI1 coupling and SOCE activation ^{22,23}. We showed that all cells displayed functional SOCE (FIG. 1F). In the presence of the ORAI channel blocker synta-66²⁴, SOCE in HSC-3 and SCC-9 cells was significantly reduced (FIG. 1G). We also examined the contribution of other channels that have been implicated in Ca²⁺ influx in various cancer cells. Stimulation of transient receptor channels TRPM7, TRPA1 and TRPV1 in HSC-3 cells using the specific agonists naltriben, AITC or capsaicin, respectively, in the presence of Ca²⁺, failed to evoke an increase in cytosolic Ca²⁺.

Molecular Knockdown of ORAI1 Reduces Ca²⁺ Influx and Cell Proliferation

To address the role of SOCE in key cancer hallmarks such as cell proliferation in oral cancer, we used two independent approaches to eliminate ORAI1 expression in HSC-3 cells. Lentiviral knockdown of ORAI1 by shRNA in HSC-3 cells reduced SOCE by ˜70% (FIG. 1, H and I). Similar results were obtained in shORAI1 SCC-9, DOK and HaCaT cells. Because the ORAI blocker synta-66 abolished SOCE, but ORAI1 shRNA only reduced it, this observation raised the possibility that the ORAI2 or ORAI3 proteins may enable residual Ca²⁺ influx given that the ORAI channel is formed by heteromers of the three ORAI subunits^{23,25,26}. To resolve this issue and given the similar responses of both HSC-3 and SCC-9 cells, we used CRISPR/Cas9 to delete ORAI1 only in HSC-3 cells and found SOCE to be abolished (FIG. 1, J and K). These data indicate that ORAI1 was key in mediating SOCE in oral cancer cells. To address the effects of ORAI1 in cell proliferation, we analyzed HSC-3 cells in the presence of synta-66 and in which ORAI1 expression had been reduced by shORAI1 or CRISPR/Cas9 editing using the colorimetric assay MTS (FIG. 1, L and M). In this assay, proliferation was significantly decreased compared to control cells. However, ORAI1 deletion did not induce cell death.

MMP-1 Expression is Upregulated by SOCE Activation and is Mediated Via NFAT

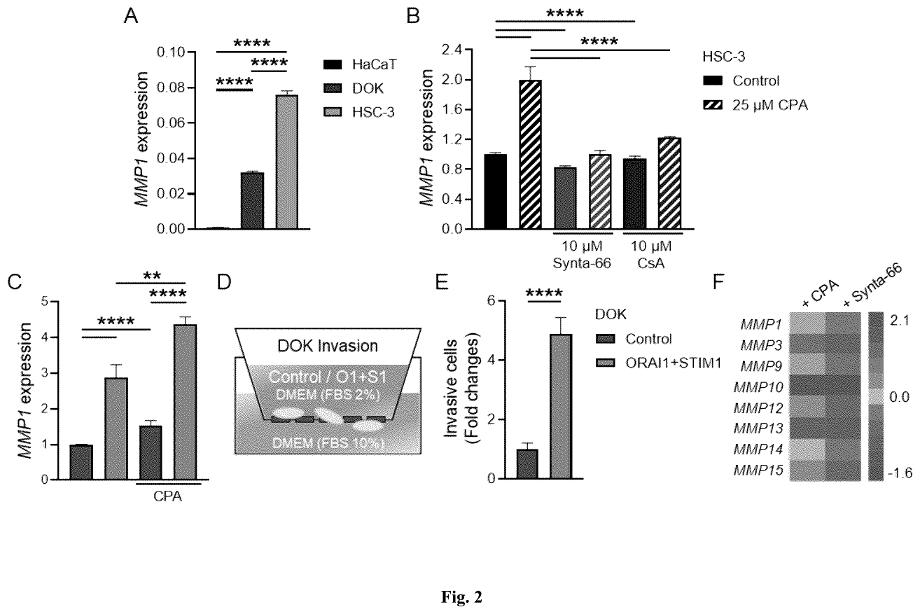

The role of ORAI1 in oral cancer progression was further elucidated by examining the expression of oncogenes in oral cancer, such as MMP1^{9,27}, as a proof of concept. We first verified the expression of MMP1 in HaCaT, DOK and HSC-3 cells by qRT-PCR analysis. All cells expressed MMP1 in the order oral cancer HSC-3 cells>dysplastic DOK cells>HaCaT cells (FIG. 2A). We next stimulated HSC-3 cells using different concentrations of CPA to examine changes in MMP1 gene expression mediated by SOCE activation. We found that 25 μM CPA elicited a significant increase in MMP1 in HSC-3 cells, with a maximal effect seen at 3 hours of CPA stimulation. Blocking ORAI with synta-66 attenuated the increase in MMP1 gene expression, suggesting that MMP1 gene expression was modulated by SOCE (FIG. 2B). These findings were further supported by the decrease in the expression of MMP1 seen in HSC-3 cells with ORAI1 knockdown or deletion. Because in many cells SOCE activation targets the phosphorylation of the transcription factor NFAT (nuclear factor of activated T cells) and its downstream effectors ²⁸, and because a previous study implicated NFAT in regulating stemness in oral cancer cells ¹⁶, we next interrogated if the transcriptional up-regulation of MMP1 was downstream of NFAT. We first verified the expression of NFAT isoforms (NFAT1-4) in HaCaT, DOK and HSC-3 cells by qRT-PCR analysis showing that NFAT4 (coded by NFATc3) was the most abundant transcript. We showed that inhibition of NFAT using cyclosporine A (CsA) in HSC-3 cells reduced the increase in MMP1 expression elicited by CPA (FIG. 2B). Similar results were identified in DOK cells treated with CsA. Collectively, these data demonstrate that MMP1 gene expression is controlled by SOCE presumably through a direct or indirect involvement of NFAT.

SOCE Enhances MMP1 Expression and Migration of Dysplastic Oral Keratinocytes In Vitro.

We next evaluated whether SOCE was involved in the progression from oral dysplasia to cancer by assessing the effect of SOCE on the expression of MMP1 in DOK cells. We overexpressed ORAI1 and STIM1 in DOK cells in a 1:2 ratio based on a previous report ²⁹. We first analyzed the expression of ORAI1 in the transfected DOK cells which showed a significant up-regulation of ORAI1 mRNA. We then analyzed MMP1 gene expression showing that overexpressing STIM1 and ORAI1 alone increased MMP1 expression in DOK cells, which was further enhanced following the activation of SOCE by CPA (FIG. 2C). To determine if these changes in MMP1 gene expression in DOK cells, driven by SOCE, elicited changes in cell behavior to become invasive, we analyzed cell invasion using Matrigel-coated Boyden chambers ³⁰. DOK cells are non-invasive cells, but DOK cells overexpressing STIM1 and ORAI1 exhibited increased ability to migrate through the ECM in vitro (FIG. 2, D and E). These data support the notion that SOCE is involved in the transformation of oral dysplasia to cancer by enhancing the expression of key biomarkers and altering the behavior of non-cancer dysplastic cells.

SOCE Stimulation of HSC-3 Cells Treated with ORAI Blocker Show Bulk Down-Regulation of MMPs and Pain Modulators

To address global changes in gene expression evoked by the activation of ORAI1, we performed a bulk RNA sequencing (RNA-seq) of the oral cancer HSC-3 cells. ORAI1 was activated using CPA in the presence or absence of the ORAI inhibitor synta-66. Differential gene expression analysis was performed and heatmaps generated to show differentially expressed genes (FDR<0.05) in CPA stimulated cells in the presence or absence of synta-66. Because several MMPs are recognized as markers in the transition from dysplasia to oral cancer ^{8,9}, we first analyzed expression changes in these genes. MMPs contribute to the initiation of cancers in many epithelial cancers by degrading the basement membrane facilitating the invasion of neoplastic cells into the connective tissue 9. This step is considered a risk factor in the progression to malignancy. Our analysis of these data revealed bulk down-regulation of MMP genes with 8 out of >21 genes in the MMP family, including MMP1, being significantly down-regulated (FIG. 2F) by the ORAI inhibitor synta-66. These data confirmed the role of SOCE in the regulation of MMPs. The identification of MMP1 is particularly interesting as this gene has not been linked with pain, whereas other MMPs such as MMP2 or MMP9 have been associated with pain in previous studies ³¹. Because pain is an important feature of oral cancer patients, we also analyzed whether common markers linked to pain were differentially expressed. We showed that several pain modulators were down-regulated at the mRNA level in cells treated with the ORAI blocker including mRNAs encoding vascular endothelial growth factors (VEGF-A, B and C), NGF (nerve growth factor), TLR2 (toll-like receptor 2) and PTGS2 (prostaglandin-endoperoxide synthase 2), all of which have been implicated in pain ³²⁻³⁶.

ORAI1 Knockdown Decreased Tumor Growth In-Vivo

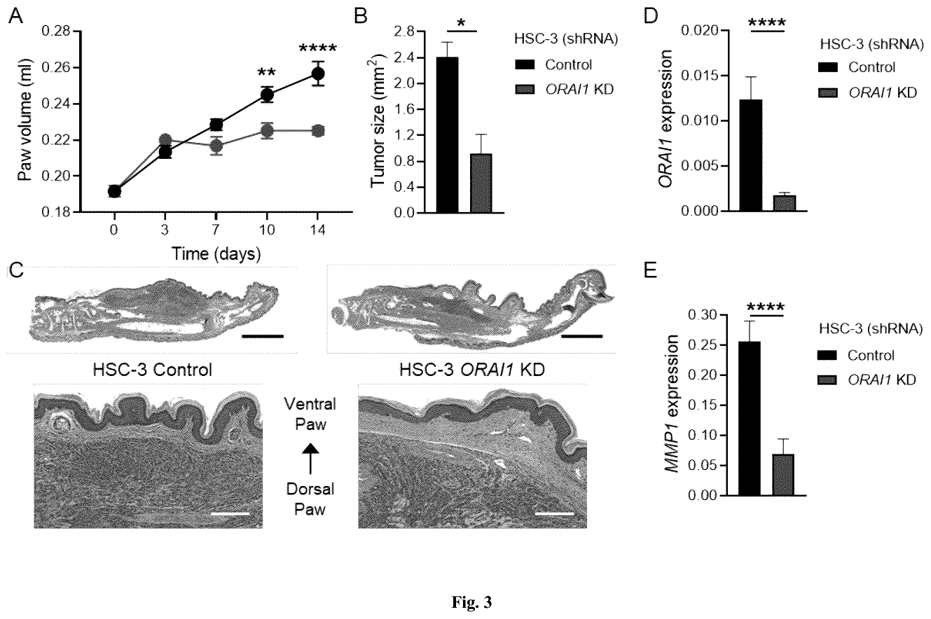

As shown above, loss of ORAI1 decreases the proliferation of oral cancer cells. We therefore hypothesized that loss of ORAI1 should attenuate tumor growth in vivo which we addressed by analyzing the growth of tumors formed by human shORAI1 HSC-3 cells inoculated into athymic nude mice. We observed that paw volume in mice injected with shORAI1 HSC-3 cells did not increase at day 3 and 7, whereas paw volume increased with time in paws injected with control HSC-3 cells and was significantly greater compared to shORAI1 HSC-3 inoculated paws at day 10 and 14 (FIG. 3A). These findings were further validated by analysis of tissue sections following H&E staining showing decreased tumor size in shORAI1 HSC-3 inoculated paws (FIGS. 3, B and C). The expression of ORAI1 was also significantly down-regulated in paw tissues from mice inoculated with shORAI1 HSC-3 cells (FIG. 3 D). We also analyzed MMP1 levels because of its role in oral cancer progression and as a potential pain modulator and showed that its expression was also significantly down-regulated (FIG. 3 E), supporting a link between ORAI1 and MMP 1.

SOCE Stimulation of HSC3 Cells Treated with ORAI1 and NFAT Blockers Show Bulk Downregulation of MMP and Pain Markers

Loss of ORAI1 in Murine MOC-2 Oral Cancer Cells Attenuates Allodynia In-Vivo

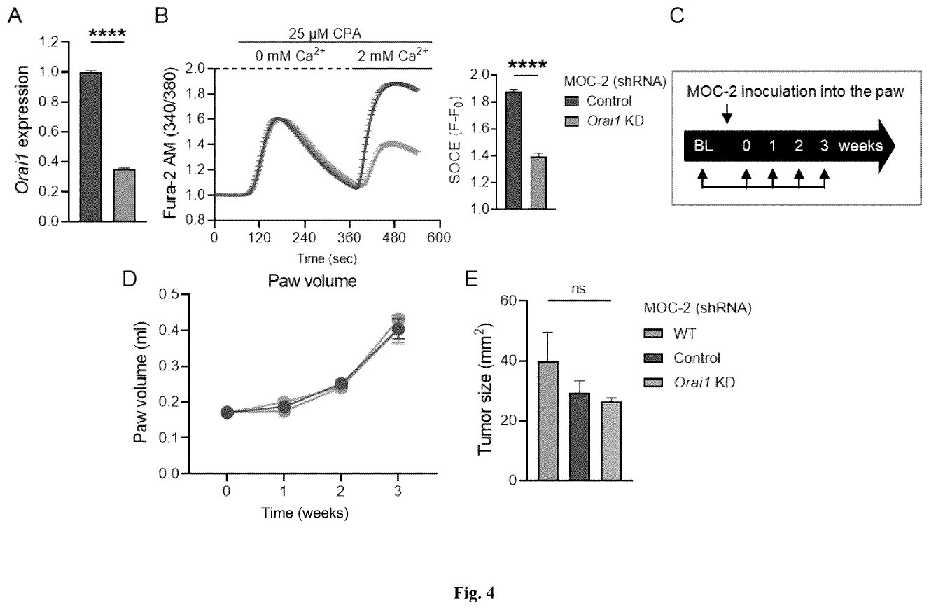

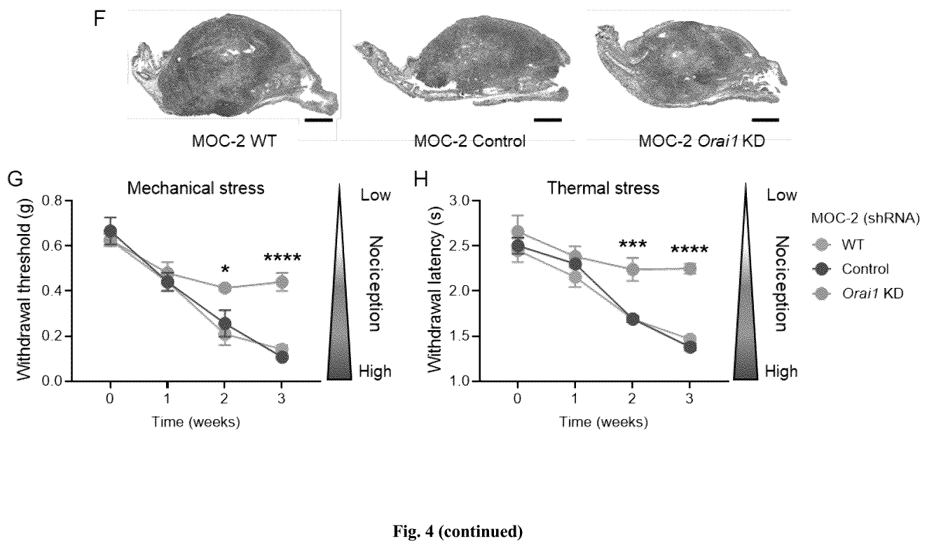

Because pain is a chief complaint of oral cancer patients and because we found that blocking ORAI1 down-regulated several cancer pain related genes, we next addressed whether loss of Orai1 could influence mechanical and thermal sensitivities in vivo. First, we quantified Ca²⁺ dynamics in the murine oral cancer MOC-2 cells with Orai1 knockdown, which showed significantly diminished Ca²⁺ influx compared to control cells (FIGS. 4, A and B). We next inoculated wildtype (WT), vehicle (Control), and shOrai1 (Orai1 KD) MOC-2 cells into the center of the left plantar of C57BL/6J WT male mice to analyze sensitization to mechanical (von Frey) and thermal (infrared light) noxious stimuli over 3-weeks (FIG. 4C). We chose this paw xenograft model because it is easy to measure the paw volume using the Plethysmometer by displacing a measuring fluid when the paw is immersed, indicating swelling, and because it has been widely used in oral cancer models linked to pain³⁷⁻³⁹. This single-blind experiment showed that although Orai1 knockdown did not show differences across groups in the growth of MOC-2 allografts (FIG. 4, D-F), mice inoculated with shOrai1 MOC-2 cells showed a significant decrease in hypersensitivity to both mechanical and thermal stimuli at weeks 2 and 3 after the inoculation (FIG. 4, G and H). These data suggest the involvement of ORAI1 in cancer pain in vivo.

Loss of ORAI1 Attenuates Excitation of Trigeminal Neurons

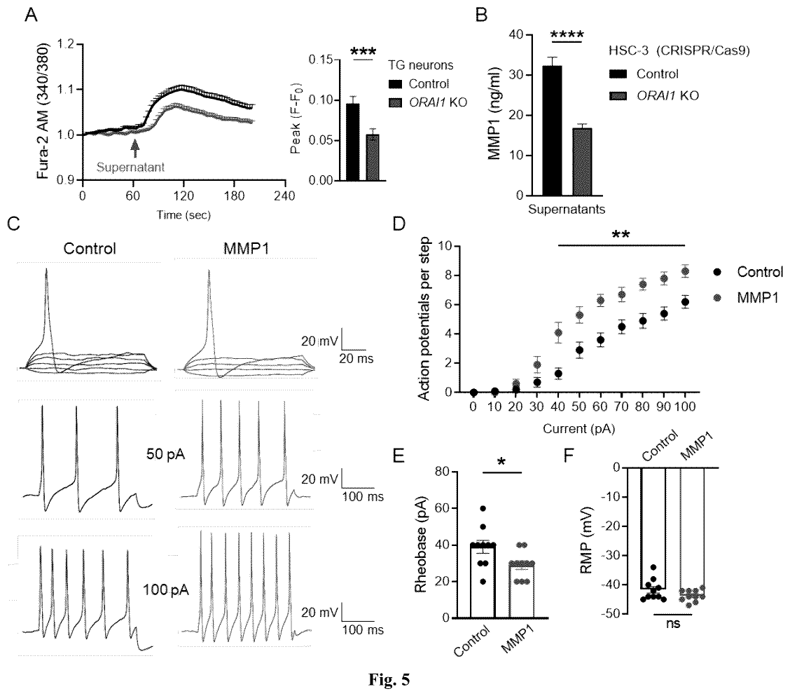

The decrease in mechanical and thermal hypersensitivities evoked by cancer cells lacking ORAI1 might indicate an attenuation in pain transmission. Oral cancer cells release nociceptive mediators that activate and sensitize primary afferent neurons causing pain. In the oral region, pain sensation is carried by the trigeminal pathway to the brain. Because measuring changes in cytosolic Ca²⁺ concentration ([Ca²⁺]_{cyto}) in neurons is a robust proxy to determine changes in excitability⁴⁰, we loaded neurons from the trigeminal ganglia (TG) of WT mice with the cytosolic Ca²⁺ indicator Fura2-AM and stimulated them with the supernatant conditioned by HSC-3 cells with a CRISPR/Cas9 deletion of ORAI Ca²⁺ influx in TG neurons incubated with the supernatant of ORAI1-deficient HSC-3 cells was reduced compared to HSC-3 cells with intact ORAI1 (FIG. 5A). Because we showed that lack of ORAI1 in HSC-3 cells decreased the mRNA expression of MMP1, we asked if the secreted levels of MMP1 might also be reduced in the supernatant of HSC-3 cells lacking ORAI1. Indeed, ELISA showed that cells lacking ORAI1 contained significantly lower levels of MMP1 compared to control cells (FIG. 5B). These data indicate that ORAI1 regulates nociceptive mediators released by the oral cancer cells that modulate the excitation of TG neurons and suggest that MMP1 might be one of the nociceptive mediators involved in pain transmission. To further address if MMP1 is directly involved in neuronal excitation, we used whole-cell patch clamp to record action potentials in TG neurons stimulated with an activated MMP1 peptide. In the presence of MMP1, TG neurons exhibited increased numbers of action potentials (˜30% increase) (FIG. 5, C and D). MMP1 stimulation resulted in lowered rheobase (minimal current needed to elicit an action potential) without influencing the resting membrane potential (FIG. 5, E and F), suggesting that MMP1 increases TG excitability and thus supporting a role of MMP1 in pain transmission.

Discussion

MMPs are Ca²⁺ dependent enzymes involved in the degradation of ECM. Overexpression of several MMPs, including MMP-1, has been associated with tumor invasion and correlate with poor prognosis. We have previously shown that MMP-1 mediates the progression from dysplasia to cancer ⁹. While not all dysplastic lesions will progress to malignancy, an abundance of MMP-1 is an indication of this change, as their proteolytic activity over membranes of adjacent tissues enables invasion. In quiescent conditions, MMP expression is low. Their transcriptional activation is unclear with several molecules, such as tumor necrosis factor (TNF) and interleukin (IL)-1, involved in the overexpression of MMPs ⁴¹. IL-1 processing depends and its release is dependent on calcium/calmodulin interactions ⁴². Such an interaction is also an essential step in the activation of NFAT by SOCE.

The present disclosure addresses the question of whether SOCE is involved in oral cancer progression and pain. We show that tissues from patients with tongue cancer have significantly higher expression of ORAI1 and ORAI2, analyzed by RNASeq, than healthy controls.

Several MMPs have been recognized as markers in the transition from oral dysplasia to cancer and are upregulated in tumors from oral cancer patients. Our data shows that MMP expression is regulated by SOCE and more specifically, are downstream of the transcription factor NFAT. This is also the case in atrial myocytes where NFAT is known to regulate MMP-1 expression.

### REFERENCES

1 Prevarskaya, N., Skryma, R. & Shuba, Y. Ion Channels in Cancer: Are Cancer Hallmarks Oncochannelopathies? Physiol Rev 98, 559-621, doi:10.1152/physrev.00044.2016 (2018).
2 Cui, C., Merritt, R., Fu, L. & Pan, Z. Targeting calcium signaling in cancer therapy. Acta Pharm Sin B 7, 3-17, doi:10.1016/j.apsb.2016.11.001 (2017).
3 Chalmers, S. B. & Monteith, G. R. ORAI channels and cancer. Cell Calcium 74, 160-167, doi:10.1016/j.ceca.2018.07.011 (2018).
4 Monteith, G. R., Prevarskaya, N. & Roberts-Thomson, S. J. The calcium-cancer signalling nexus. Nat Rev Cancer 17, 367-380, doi:10.1038/nrc.2017.18 (2017).
5 Kiss, F., Pohoczky, K., Szallasi, A. & Helyes, Z. Transient Receptor Potential (TRP) Channels in Head-and-Neck Squamous Cell Carcinomas: Diagnostic, Prognostic, and Therapeutic Potentials. Int J Mot Sci 21, doi:10.3390/ijms21176374 (2020).
6 Quintero-Fabian, S. et al. Role of Matrix Metalloproteinases in Angiogenesis and Cancer. Front Oncol 9, 1370, doi:10.3389/fonc.2019.01370 (2019).
7 Page-McCaw, A., Ewald, A. J. & Werb, Z. Matrix metalloproteinases and the regulation of tissue remodelling. Nat Rev Mol Cell Biol 8, 221-233, doi:10.1038/nrm2125 (2007).
8 Stott-Miller, M. et al. Tumor and salivary matrix metalloproteinase levels are strong diagnostic markers of oral squamous cell carcinoma. Cancer Epidemiol Biomarkers Prev 2628-2636, doi:10.1158/1055-9965.EPI-11-0503 (2011).
9 Jordan, R. C. et al. Overexpression of matrix metalloproteinase-1 and -9 mRNA is associated with progression of oral dysplasia to cancer. Clin Cancer Res 10, 6460-6465, doi:10.1158/1078-0432.CCR-04-0656 (2004).
10 Lacruz, R. S. & Feske, S. Diseases caused by mutations in ORAI1 and STIM1. Ann N Y Acad Sci 1356, 45-79, doi:10.1111/nyas.12938 (2015).
11 Prakriya, M. & Lewis, R. S. Store-Operated Calcium Channels. Physiol Rev 95, 1383-1436, doi:10.1152/physrev.00020.2014 (2015).
12 Yeh, Y. C. & Parekh, A. B. in Calcium Entry Channels in Non-Excitable Cells (eds J. A. Kozak & J. W. Putney, Jr.) 93-106 (2018).
13 Tiffner, A. & Derler, I. Molecular Choreography and Structure of Ca(2+) Release-Activated Ca(2+) (CRAC) and KCa2+ Channels and Their Relevance in Disease with Special Focus on Cancer. Membranes (Basel) 10, doi:10.3390/membranes10120425 (2020).
14 Prevarskaya, N., Skryma, R. & Shuba, Y. Ion channels and the hallmarks of cancer. Trends Mol Med 16, 107-121, doi:10.1016/j.molmed.2010.01.005 (2010).
15 Prevarskaya, N., Skryma, R. & Shuba, Y. Targeting Ca(2)(+) transport in cancer: close reality or long perspective? Expert Opin Ther Targets 17, 225-241, doi:10.1517/14728222.2013.741594 (2013).
16 Lee, S. H. et al. Orai1 promotes tumor progression by enhancing cancer stemness via NFAT signaling in oral/oropharyngeal squamous cell carcinoma. Oncotarget 7, 43239-43255, doi:10.18632/oncotarget.9755 (2016).
17 Singh, A. K. et al. Orai-1 and Orai-2 regulate oral cancer cell migration and colonisation by suppressing Akt/mTOR/NF-kappaB signalling. Life Sci 261, 118372, doi:10.1016/j.lfs.2020.118372 (2020).
18 Wang, Y. Y. et al. Expression of Orai1 and STIM1 in human oral squamous cell carcinogenesis. J Dent Sci 17, 78-88, doi:10.1016/j.jds.2021.07.004 (2022).
19 Dou, Y. et al. Orai1 Plays a Crucial Role in Central Sensitization by Modulating Neuronal Excitability. J Neurosci 38, 887-900, doi:10.1523/JNEUROSCI.3007-17.2017 (2018).
20 Gao, R. et al. Potent analgesic effects of a store-operated calcium channel inhibitor. Pain 154, 2034-2044, doi:10.1016/j.pain.2013.06.017 (2013).
21 Bhattacharya, A. et al. Oncogenes overexpressed in metastatic oral cancers from patients with pain: potential pain mediators released in exosomes. Sci Rep 10, 14724, doi:10.1038/541598-020-71298-y (2020).
22 Aulestia, F. J. et al. Fluoride exposure alters Ca(2+) signaling and mitochondrial function in enamel cells. Sci Signal 13, doi:10.1126/scisignal.aay0086 (2020).
23 Eckstein, M. et al. Differential regulation of Ca(2+) influx by ORAI channels mediates enamel mineralization. Sci Signal 12, doi:10.1126/scisignal.aav4663 (2019).
24 Jairaman, A. & Prakriya, M. Molecular pharmacology of store-operated CRAC channels. Channels (Austin) 7, 402-414, doi:10.4161/chan.25292 (2013).
25 Vaeth, M. et al. ORAI2 modulates store-operated calcium entry and T cell-mediated immunity. Nat Commun 8, 14714, doi:10.1038/ncomms14714 (2017).
26 Yoast, R. E. et al. The native ORAI channel trio underlies the diversity of Ca(2+) signaling events. Nat Commun 11, 2444, doi:10.1038/s41467-020-16232-6 (2020).
27 George, A., Ranganathan, K. & Rao, U. K. Expression of MMP-1 in histopathological different grades of oral squamous cell carcinoma and in normal buccal mucosa—an immunohistochemical study. Cancer Biomark 7, 275-283, doi:10.3233/CBM-2010-0191 (2010).
28 Feske, S. et al. A mutation in Orai1 causes immune deficiency by abrogating CRAC channel function. Nature 441, 179-185, doi:10.1038/nature04702 (2006).
29 Hoover, P. J. & Lewis, R. S. Stoichiometric requirements for trapping and gating of Ca2+ release-activated Ca2+ (CRAC) channels by stromal interaction molecule 1 (STIM1). Proc Natl Acad Sci USA 108, 13299-13304, doi:10.1073/pnas.1101664108 (2011).
30 Salvo, E., Saraithong, P., Curtin, J. G., Janal, M. N. & Ye, Y. Reciprocal interactions between cancer and Schwann cells contribute to oral cancer progression and pain. Heliyon 5, e01223, doi:10.1016/j.heliyon.2019.e01223 (2019).
31 Kuhad, A., Singh, P. & Chopra, K. Matrix metalloproteinases: potential therapeutic target for diabetic neuropathic pain. Expert Opin Ther Targets 19, 177-185, doi:10.1517/14728222.2014.960844 (2015).
32 Selvaraj, D. et al. A Functional Role for VEGFR1 Expressed in Peripheral Sensory Neurons in Cancer Pain. Cancer Cell 27, 780-796, doi:10.1016/j.ccell.2015.04.017 (2015).
33 Bimonte, S., Cascella, M., Forte, C. A., Esposito, G. & Cuomo, A. The Role of Anti-Nerve Growth Factor Monoclonal Antibodies in the Control of Chronic Cancer and Non-Cancer Pain. J Pain Res 14, 1959-1967, doi:10.2147/JPR.S302004 (2021).
34 Lacagnina, M. J., Watkins, L. R. & Grace, P. M. Toll-like receptors and their role in persistent pain. Pharmacol Ther 184, 145-158, doi:10.1016/j.pharmthera.2017.10.006 (2018).
35 Llorian-Salvador, M. & Gonzalez-Rodriguez, S. Painful Understanding of VEGF. Front Pharmacol 9, 1267, doi:10.3389/fphar.2018.01267 (2018).
36 Rausch, S. M. et al. SNPs in PTGS2 and LTA predict pain and quality of life in long term lung cancer survivors. Lung Cancer 77, 217-223, doi:10.1016/j.lungcan.2012.02.017 (2012).
37 Ye, Y. et al. Nerve growth factor links oral cancer progression, pain, and cachexia. Mol Cancer Ther 10, 1667-1676, doi:10.1158/1535-7163.MCT-11-0123 (2011).
38 Tu, N. H. et al. Legumain Induces Oral Cancer Pain by Biased Agonism of Protease-Activated Receptor-2. J Neurosci 41, 193-210, doi:10.1523/JNEUROSCI.1211-20.2020 (2021).
39 Tu, N. H. et al. Cathepsin S Evokes PAR2-Dependent Pain in Oral Squamous Cell Carcinoma Patients and Preclinical Mouse Models. Cancers (Basel) 13, doi:10.3390/cancers13184697 (2021).
40 Berridge, M. J. Neuronal calcium signaling. Neuron 21, 13-26, doi:10.1016/s0896-6273(00)80510-3 (1998).
41 Loffek, S., Schilling, O. & Franzke, C. W. Series “matrix metalloproteinases in lung health and disease”: Biological role of matrix metalloproteinases: a critical balance. Eur Respir J 38, 191-208, doi:10.1183/09031936.00146510 (2011).
42 Ainscough, J. S., Gerberick, G. F., Kimber, I. & Dearman, R. J. Interleukin-1beta Processing Is Dependent on a Calcium-mediated Interaction with Calmodulin. J Biol Chem 290, 31151-31161, doi:10.1074/jbc.M115.680694 (2015).

Although the present disclosure has been described with respect to one or more particular embodiments and/or examples, it will be understood that other embodiments and/or examples of the present disclosure may be made without departing from the scope of the present disclosure.

1. A method comprising administering to an individual who has oral cancer a composition comprising a calcium channel blocker.

2. The method of claim 2, wherein the calcium channel blocker is specific for ORAI Ca²⁺ channels.

3. The method of claim 2, wherein the calcium channel blocker is selected from the group consisting of synta66, BTP-2 (YM-58483), CM-2489, CM-4620, GSK-5498A, GSK-7975A, and a combination thereof.

4. The method of claim 3, wherein the calcium channel blocker is CM-4620.

5. The method of claim 3, wherein administering the composition comprising the calcium channel blocker inhibits growth of the oral cancer, or inhibits development of allodynia in the individual.

6. The method of claim 5, wherein inhibiting the growth of the oral cancer comprises inhibiting transformation of oral keratinocytes to a squamous cell carcinoma.

7. The method of claim 5, wherein administering the composition comprising the calcium channel blocker inhibits development of allodynia in the individual.
